(19) United States
(12) Patent Application Publication
(10) Pub. No.: US 2026/0083667 A1
(43) Pub. Date: Mar. 26, 2026

(54) SULFORAPHANE SUPPLEMENT IN GUMMY FORM

(71) Applicant: Sensory Scout LLC, St. Petersburg, FL (US)

(72) Inventor: Benzion Sadigursky, St. Petersburg, FL (US)

(73) Assignee: Sensory Scout LLC, St. Petersburg, FL (US)

(21) Appl. No.: 19/341,677

(22) Filed: Sep. 26, 2025

Related U.S. Application Data
(60) Provisional application No. 63/699,705, filed on Sep. 26, 2024.

(51) Int. Cl. A61K 9/00; A61K 31/26; A61K 31/7028; A61K 36/31; A61K 38/47; A61K 47/12; A61K 47/26; A61K 47/36

(52) U.S. Cl. CPC A61K 9/0056; A61K 31/26; A61K 31/7028; A61K 36/31; A61K 38/47; A61K 47/12; A61K 47/26; A61K 47/36; C12Y 302/01147

(57) ABSTRACT

A sulforaphane supplement in gummy form, including an active ingredient composition comprising sulforaphane, glucoraphanin, and myrosinase. The supplement including a matrix structure in which sulforaphane, glucoraphanin, and myrosinase are disposed in pores and immobilized therein.

### CROSS-REFERENCE TO RELATED APPLICATIONS

This application claim the benefit of U.S. Provisional Patent Application Ser. No. 63/699,705, filed on Sep. 26, 2024, and titled “SULFORAPHANE SUPPLEMENT IN GUMMY FORM”, which is incorporated by reference herein in its entirety.

### BACKGROUND

Sulforaphane is a naturally occurring isothiocyanate compound found primarily in cruciferous vegetables such as broccoli, brussels sprouts, and kale. In the plant, sulforaphane does not exist directly but is produced when its precursor glucoraphanin (a glucosinolate) is hydrolyzed by the enzyme myrosinase. This reaction typically occurs when plant tissues are damaged (e.g., during chewing, cutting, or crushing), allowing myrosinase and glucoraphanin to come into contact and produce sulforaphane.

Sulforaphane has gained attention for its potential health benefits, including anti-inflammatory properties and anticancer effects. As an active ingredient in dietary supplements, sulforaphane is often marketed to promote overall wellness and disease prevention. Further, research suggests that sulforaphane may have beneficial impacts on such neurodevelopmental disorders. Early research indicates that sulforaphane could modulate cellular signaling pathways involved in neuroinflammation, oxidative stress, and mitochondrial function-all factors implicated in ASD development and progression. In addition to autism, sulforaphane has also been investigated in other neurodevelopmental disorders, such as attention deficit hyperactivity disorder (ADHD).

Palatability, taste, texture, and an overall positive sensory experience is particularly important in the context of autism spectrum disorders (ASD) and related neurodevelopmental disorders. Medication formulations for pediatric and/or patients having ASD or other neurodevelopmental disorders which provide a positive sensory experience, such as gummies, are crucial. Such formulations encourage adherence to medication regimens by making the process more enjoyable. Children and individuals on the autism spectrum often respond better to treatments that are less intimidating or stressful. These sensory-friendly forms can reduce anxiety associated with taking pills, which may be overwhelming due to their size, taste, or texture. Thus, these formulations aid in the successful management of symptoms by ensuring the medication is taken consistently and correctly, leading to better health outcomes overall.

Accordingly, there is a need for palatable formulations for sulforaphane supplementation. Such formulations have not conventionally been available, with sulforaphane or its precursors (glucoraphanin and/or myrosinase) typically being formulated as pills or capsules. Achieving an effective dosage level while ensuring palatability and ease of consumption is crucial for consumer acceptance and compliance with the supplement's intended use.

Additionally, there is a need for formulations which can provide sufficient doses of active and bioavailable sulforaphane. Commonly, sulforaphane supplements use sulforaphane itself as the active ingredient in their formulation (as opposed to the combination of its precursors glucoraphanin and myrosinase, found naturally in plant sources). This may pose challenges as sulforaphane is chemically unstable and prone to degradation due to its reactive isothiocyanate group. It is sensitive to heat, light, moisture, and oxygen, which can lead to rapid breakdown during storage, processing, or transport. This instability complicates the production, formulation, and delivery of sulforaphane supplements or functional foods, resulting in variable potency or loss of efficacy. Stabilizing sulforaphane for supplementation conventionally requires encapsulation, refrigeration, or formulation with stabilizing agents to reduce degradation and preserve bioactivity. All of these conventional methods involve additional expense (in the instance of encapsulation or addition of stabilizing agents) and/or complications to the consumer and supply chain (e.g., in the case of refrigeration).

Although some supplements attempt to deal with these stability issues by supplementing with glucoraphanin and myrosinase, without the proper formulation, this strategy may result in unreliable generation of the required sulforaphane dosage. For example, if glucoraphanin and myrosinase come into contact with one another in the formulation, they may react to form sulforaphane well before ingestion by a user, resulting in the same stability issues that sulforaphane itself has. On the other hand, if glucoraphanin and myrosinase are separated in the formulation, e.g., in two separate pills or capsules administered together, or separated by different layers or encapsulation within one pill or capsule, then the appropriate reaction to generate sulforaphane is reliant on the variable conditions of the digestive system. For optimal sulforaphane production, glucoraphanin and myrosinase must thoroughly mix under suitable conditions, which may not reliably happen in the digestive tract. The acidic environment of the stomach can denature myrosinase before it has a chance to act, further impeding sulforaphane formation. Sulforaphane yield may also depend on gut microbiota, which can partially compensate by converting glucoraphanin, but with inconsistent efficiency. These issues lead to unpredictable sulforaphane production and bioavailability when using conventional glucoraphanin-myrosinase supplements.

Accordingly, there is a need for formulations for sulforaphane supplementation which are not only palatable and acceptable to consumers, but which also provide stable and reliable doses of sulforaphane.

### BRIEF SUMMARY

Disclosed are gummy formulations for sulforaphane supplementation. The disclosed gummies comprise a comprehensive active ingredient mixture including both sulforaphane and its precursors glucoraphanin and myrosinase, in which the active ingredients are immobilized in a gummy matrix. The disclosed formulations also include excipients designed to place the active ingredient mixture in a state which is both stable, highly bioavailable, and palatable to patients. Specifically, the disclosed formulations may comprise a sweetener and a gelling agent sufficient to provide a gummy-type structure to the supplement. The gummies may additionally contain one or more flavoring agents. In some aspects, the gummy texture and the flavoring components of the supplement produce a positive sensory experience which is particularly well-suited to pediatric patients and/or patients diagnosed with autism or like neurological conditions.

According to some aspects, the disclosed supplement formulation, includes: an active ingredient consisting of sulforaphane, glucoraphanin, and myrosinase; an excipient including pectin, a sugar comprising sucrose, citric acid, and water; and a matrix formed by interconnected molecules of the pectin, defining a plurality of pores therebetween, wherein the sulforaphane, the glucoraphanin, and the myrosinase are disposed within the pores and immobilized therein, wherein in a weight ratio of the pectin to the water is within a range of 0.05 to 1.2, wherein a weight ratio of the citric acid to the pectin is within a range of 0.005 to 0.80, wherein the sucrose is present in an amount within a range of 8 wt % to 35 wt %, based on a total weight of the gummy formulation, and wherein a weight ratio of the glucoraphanin to the pectin is within a range of 0.001 to 0.2.

According to some aspects, the pectin is included in an amount within a range of 1.5 wt % and 14 wt %, or within the range of 3 wt % to 8 wt %, based on the total weight of the gummy formulation.

According to some aspects, the sulforaphane is included in an amount within a range of 0.001 wt % to 0.04 wt %, or within the range of 0.007 wt % to 0.02 wt %, based on a total weight of the gummy formulation. According to some aspects, the sugar may further include glucose. In some embodiments, a weight ratio of the sucrose to the glucose is within a range of 0.15 to 0.55.

According to some aspects, the disclosed supplement formulation may include: an active ingredient consisting of sulforaphane, glucoraphanin, and myrosinase, the formulation including a white mustard seed powder comprising the myrosinase; an excipient comprising pectin, a sugar, citric acid, and water; and a matrix formed by interconnected molecules of the pectin, defining a plurality of pores therebetween, wherein the sulforaphane, the glucoraphanin, and the myrosinase are disposed within the pores and immobilized therein, wherein a weight ratio of the white mustard seed powder to the glucoraphanin is within a range of 0.001 to 1, wherein a weight ratio of the glucoraphanin to the pectin is within a range of 0.001 to 0.2, and wherein a weight ratio of the white mustard seed powder to the pectin is within a range of 0.001 to 0.9.

According to some aspects, the weight ratio of the white mustard seed powder to the glucoraphanin is within the range of 0.01 to 0.50, or within the range of 0.02 to 0.10. According to some aspects, the weight ratio of the white mustard seed powder to the pectin is within the range of 0.005 to 0.7, or within the range of 0.2 to 0.4. According to some aspects, the weight ratio of the glucoraphanin to the pectin is within a range of 0.005 to 0.10, or within a range of 0.008 to 0.01.

According to some aspects, the present disclosure provides a method of supplementation, including: administering the disclosed supplement formulation to a user, instructing the user to chew the supplement formulation, initiating a reaction of the myrosinase and the glucoraphanin in the user's mouth. In some instances, the user is a pediatric patient who has been diagnosed with a neurological disorder. According to some embodiments, the method may additionally include administering one unit of the supplement formulation, twice per day. According to some embodiments, the method may also include a first administration of one unit of the supplement formulation and a second administration of one unit of the supplement formulation, the first and second administration being separated by an interval within a range of 8 to 14 hours.

### TERMS AND DEFINITIONS

As used in the specification and the appended claims, the singular forms “a,” “an,” “the” and the like include plural referents unless the context clearly dictates otherwise. Also, while reference may be made herein to quantitative measures, values, geometric relationships or the like, unless otherwise stated, any one or more if not all of these may be absolute or approximate to account for acceptable variations that may occur, such as those due to engineering tolerances or the like.

As used herein, a “gummy” refers to a formulation with a bendable, pliable form which bounces back when compressed or bent lightly and has a chewy texture, e.g. a chewy candy, gum drop, jelly bean, etc.

As used herein, “pediatric” refers to patients and individuals under the age of 18, for example, infants, children, and adolescents, and the health and medical care related to such patients.

As used herein, “natural” refers to substances, mixtures, or ingredients derived from natural sources (e.g. Plants, animals, or minerals) without significant alteration to their chemical structure(s) through synthetic processes. “Natural” components maintain their original chemical composition and properties.

As used herein, “artificial” is a synonym of “synthetic” and refers to substances, mixtures, or ingredients that are either produced by man-made processes or are subject to processing which alters their chemical structure(s) and/or chemical properties from those occurring in nature.

As used herein, unless otherwise defined, “active ingredient” refers to the component in a product, such as a drug or supplement, that is directly biologically or chemically responsible for its intended therapeutic or functional effect. In other words, it is the substance that produces the desired physiological or pharmacological action, distinguishing it from inactive components (excipients) that serve as carriers or stabilizers without direct biological activity.

As used herein, unless otherwise defined, “excipient” refers to a component included in a pharmaceutical formulation or supplement which does not directly contribute to the intended therapeutic or functional effect. The excipient may facilitate the correct physical and chemical properties of the final product without having direct biological activity.

### DETAILED DESCRIPTION

Disclosed are gummy formulations for sulforaphane supplementation. The disclosed gummy formulation includes a comprehensive active ingredient mixture including both sulforaphane and its precursors glucoraphanin and myrosinase. The disclosed formulations also include excipients designed to place the active ingredient mixture in a state which is both stable, highly bioavailable, and palatable to patients. Specifically, the disclosed formulations may comprise a sweetener and a gelling agent sufficient to provide a gummy-type structure to the supplement. The gummies may additionally contain one or more flavoring agents. In some aspects, the gummy texture and the flavoring components of the supplement produce a positive sensory experience which is particularly well-suited to pediatric patients and/or patients diagnosed with autism or like neurological conditions.

The structure of the disclosed formulation may include a matrix formed at least primarily by the gelling agent, which immobilizes the active ingredients separately and does not allow them to migrate or contact one another to react, prior to ingestion by a user. Because the gummy formulation may contain sweeteners, flavorants, and a gummy texture all designed to encourage a user to chew rather than to swallow the formulation, the matrix may be broken down during the chewing process, allowing the glucoraphanin and myrosinase precursors to contact one another and begin to react in the user's mouth such that sulforaphane begins to be formed before entering the more variable conditions of the digestive tract.

Active Ingredient Conventionally, sulforaphane supplements may contain either sulforaphane itself or the precursors of sulforaphane-glucoraphanin and myrosinase. Incorporating sulforaphane itself may directly into a supplement may be advantageous in that it can offer enhanced bioavailability and may have improved absorption and utilization in the body compared to its precursors. However, sulforaphane in its whole form is not highly stable and may degrade when exposed to sunlight or UV radiation, heat, and/or oxygen.

The present supplement may contain a more dynamic and comprehensive active ingredient mixture which includes both sulforaphane and its precursors glucoraphanin and myrosinase. Accordingly, the present supplement can provide the increased bioavailability of whole sulforaphane, as well as avoiding any potential issues with degradation by providing an additional boost of sulforaphane generated in vivo at the point of consumption by the combination of glucophoranin and myrosinase that occurs when a patient chews the gummy, breaking down its structure and allowing the precursors to react. Because the glucoraphanin and myrosinase are disposed and immobilized in a matrix, they may be prevented from reacting to produce sulforaphane (which may then degrade) in advance of consumption by a user. At the same time, because the gummy matrix is broken down in the mouth as a user chews, the precursors begin to contact one another prior to being swallowed, allowing for reaction occur before exposure to the variable conditions of the digestive system, such as diverse microbiological environments, the high pH in the stomach, etc. In some embodiments, the active ingredient consists of sulforaphane, glucoraphanin, and myrosinase. According to some embodiments, the active ingredient may not include one or more of mushroom extract, curcumin, piperine, probiotics, or ascorbic acid. In some embodiments, the aforementioned components may be excluded from the formulation as a whole. In alternative embodiments, additional active ingredients may be included.

In some embodiments, the sulforaphane may be provided in the form of a broccoli (brassica oleracea) extract. According to some embodiments, the broccoli (brassica oleracea) extract containing sulforaphane may be present in an amount of at least 0.5 wt %, at least 1 wt %, at least 1.5 wt %, or at least 1.8 wt %, and no more than 10 wt %, no more than 8 wt %, no more than 6 wt %, no more than 4 wt %, no more than 3.5 wt %, no more than 3 wt %, no more than 2.5 wt %, or no more than 2.2 wt %, based on a total weight of the gummy formulation.

While broccoli (brassica oleracea) extract may generally contain sulforaphane, the amount of sulforaphane in the extract is inconsistent and may differ significantly based on the process and supplier used. Accordingly, it is important to control the sulforaphane levels themselves in the extract and formulation, rather than merely the amount of extract. According to some aspects, the extract may contain at least at least 0.1 wt %, at least 0.2 wt %, at least 0.3 wt %, or at least 0.35 wt %, and no more than 1 wt %, no more than 0.8 wt %, no more than 0.6 wt %, no more than 0.5 wt %, or no more than 0.45 wt % sulforaphane, based on a total weight of the extract.

Most importantly, the total amount of sulforaphane in the formulation must be controlled. According to some embodiments, the sulforaphane may be present in an amount of at least 0.001 wt %, at least 0.003 wt %, at least 0.005 wt %, or at least 0.007 wt %, and no more than 0.009 wt %, no more than 0.01 wt %, no more than 0.015 wt %, no more than 0.02 wt %, no more than 0.025 wt %, no more than 0.03 wt %, no more than 0.035 wt %, or no more than 0.04 wt %, based on a total weight of the gummy formulation. If the sulforaphane is provided in too low an amount, there may not be sufficient active and non-degraded sulforaphane left to provide any functional or therapeutic effect by the time it is consumed. On the other hand, if too much sulforaphane is included, it may cause negative side-effects such as gastrointestinal distress, when consumed.

In some embodiments, the glucophoranin may be provided in the form of a broccoli (brassica oleracea) extract. According to some embodiments, the broccoli (brassica oleracea) extract containing glucoraphanin may be present in an amount of in an amount of at least 0.5 wt %, at least 1 wt %, at least 1.5 wt %, or at least 1.8 wt %, and no more than 10 wt %, no more than 8 wt %, no more than 6 wt %, no more than 4 wt %, no more than 3.5 wt %, no more than 3 wt %, no more than 2.5 wt %, or no more than 2.2 wt %, based on a total weight of the gummy formulation.

While broccoli (brassica oleracea) extract may generally contain glucoraphanin, the amount of glucoraphanin in the extract is inconsistent and may differ significantly based on the process and supplier used. Accordingly, it is important to control the glucoraphanin levels themselves in the extract and formulation, rather than merely the amount of extract. According to some aspects, the extract may contain at least 0.3 wt %, at least 0.5 wt %, at least 0.7 wt %, or at least 0.9 wt %, and no more than 1.1 wt %, no more than 1.3 wt %, no more than 1.5 wt %, no more than 1.7 wt %, no more than 1.9 wt %, no more than 2 wt %, no more than 3 wt %, or no more than 4 wt % glucoraphanin, based on a total weight of the extract.

Most importantly, the total amount of glucoraphanin in the formulation must be controlled. According to some embodiments, the glucoraphanin may be present in an amount of at least 0.006 wt %, at least 0.008 wt %, at least 0.01 wt %, or at least 0.015 wt %, and no more than 0.025 wt %, no more than 0.03 wt %, no more than 0.035 wt %, no more than 0.04 wt %, no more than 0.045 wt %, no more than 0.05 wt %, no more than 0.055 wt %, or no more than 0.06 wt %, based on a total weight of the gummy formulation. If the glucoraphanin is provided in too low an amount, there may not be sufficient precursor available to generate enough sulforaphane in vivo to make up for any degraded whole sulforaphane, to provide a therapeutic or functional dose. On the other hand, if too much glucoraphanin is included, it may not be able to fully convert to sulforaphane, resulting in wasted ingredients and expense. Alternatively, if too large an amount of sulforaphane is generated, this may cause negative side-effects such as gastrointestinal distress.

In some embodiments, the myrosinase may be provided in the form of white mustard seed (sinapis alba L.) powder. According to some embodiments, white mustard seed (sinapis alba L.) powder may be present in an amount of at least 0.2 wt %, at least 0.4 wt %, at least 0.6 wt %, or at least 0.8 wt %, and no more than 0.9 wt %, no more than 1.1 wt %, no more than 1.3 wt %, no more than 1.5 wt %, no more than 1.7 wt %, no more than 1.9 wt %, no more than 2.5 wt %, or no more than 3 wt %, based on a total weight of the gummy formulation. According to some aspects, the white mustard seed powder and the glucoraphanin may be provided in a weight ratio of at least 0.001, at least 0.005, at least 0.01, at least 0.02, and no greater than 1.0, no greater than 0.50, no greater than 0.20, no greater than 0.10, no greater than, or no greater than 0.05. If the myrosinase is provided in too low an amount, especially in relation to the glucoraphanin, there may not be sufficient enzyme available to generate enough sulforaphane in vivo to make up for any degraded whole sulforaphane, to provide a therapeutic or functional dose. On the other hand, if too much myrosinase is included, it may be present in excess of the amount needed to convert the glucoraphanin in the formulation, resulting in wasted ingredients and expense.

### Excipient

In additional to the comprehensive active ingredient mixture, the disclosed sulforaphane supplement include an excipient or carrier. The excipient may include at least a gelling agent and a sweetener. According to some embodiments, the excipient may additionally include an acid. In some embodiments, the excipients may consist of natural materials and may not include any artificial ingredients.

The gelling agent may include be a gelatin or a pectin-based compound. In some embodiments, the gelling agent may additionally or alternatively include agar-agar, guar gum, carrageenan, xanthan gum, locust bean gum, or other known gelling agents suitable for ingestion and use in medical and culinary applications. In some embodiments, the gelling agent may consist of natural gelling agents and no artificial gelling agents may be included. In a preferred embodiment, the gelling agent is a pectin.

Pectin molecules are polysaccharides with galacturonic acid units that interact through hydrogen bonding and ionic cross-linking. Pectin forms a matrix by undergoing gelation in the presence of water and, depending on its type, specific conditions such as acidic pH and/or the presence of divalent cations (e.g., calcium ions). When hydrated, pectin chains associate to create a three-dimensional network or matrix that traps water and other components, such as the individual active ingredients, resulting in a gel or “gummy” structure. Low-methoxyl pectins gel in the presence of calcium, while high-methoxyl pectins gel under acidic conditions with sufficient sugar concentration. Appropriate formation of a matrix structure is for texture modulation and user experience, as well as for proper immobilization and associated stabilization of glucoraphanin and myrosinase.

According to some aspects, the gelling agent may be included in an amount of at least 1.5 wt %, at least 1 wt %, at least 2.5 wt %, or at least 3 wt %, and no more than 3.5 wt %, no more than 4 wt %, no more than 5 wt %, no more than 6 wt %, no more than 8 wt %, no more than 10 wt %, no more than 12 wt %, or no more than 14 wt %, based on a total weight of the gummy formulation. It is important that the gelling agent is provided in sufficient amounts, such as within the disclosed ranges, in order to produce a matrix structure. The matrix structure serves multiple functions, including providing a gummy texture which is appealing to users, providing structural stability to the formulation such that it is pliable but still freestanding, and immobilization of actives.

According to some aspects, the glucoraphanin and the gelling agent may be provided in a weight ratio of at least 0.001, at least 0.003, at least 0.005, at least 0.007, and no greater than 0.008, no greater than 0.01, no greater than 0.05, no greater than 0.10, no greater than 0.15, or no greater than 0.2. The white mustard seed powder and the gelling agent may be provided in a weight ratio of at least 0.001, at least 0.003, at least 0.005, at least 0.2, and no greater than 0.3, no greater than 0.4, no greater than 0.5, no greater than 0.7, no greater than 0.9, or no greater than 1. If the glucoraphanin and/or the white mustard seed powder (containing myrosinase) are included in too great an amount in relation to the gelling agent, they may not be sufficiently separated by the matrix, so as to prevent reaction prior to chewing by a user. Alternatively, too little of these ingredients in relation to the gelling agent may result in low active ingredient concentrations per volume, resulting in the need for a user to take many gummies to achieve the desired dosage.

Additionally, the disclosed formulation may include water. According to some embodiments, the water may be present in an amount of at least 2 wt %, at least 4 wt %, at least 6 wt %, or at least 8 wt %, and no more than 12 wt %, no more than 14 wt %, no more than 16 wt %, no more than 18 wt %, no more than 20 wt %, no more than 22 wt %, no more than 25 wt %, or no more than 30 wt %, based on a total weight of the gummy formulation. When the water is included in too great an amount, the active ingredients may not be properly immobilized. That is, the glucoraphanin and the myrosinase may be able to contact one another within the formulation and begin reaction to form sulforaphane prior to chewing by a user. In extreme cases, excess water may cause the structural stability of the formulation and the matrix to degrade. In other cases, too little water content may prevent the matrix from forming because the gelling agent, e.g., the pectin is insufficiently hydrated. In order to prevent such undesirable outcomes, the pectin and the water may be present in a weight ratio of at least 0.05, at least 0.1, at least 0.2, at least 0.3, and no greater than 0.4, no greater than 0.5, no greater than 0.7, no greater than 0.9, no greater than 1, or no greater than 1.2.

The sweetener may include a sugar comprising one or more of granulated sugar, corn syrup, glucose, sucrose, or cane sugar. The sweetener may additionally or alternatively include a sugar-free sweetener such as a sugar-alcohol, stevia, erythritol, xylitol, monk fruit extract, allulose, aspartame, sucralose, neotame, acesulfame potassayer, or thaumatin. In some embodiments, the sweeteners may consist of natural sweeteners such as glucose, cane sugar, stevia, monk fruit extract, and the like. In a preferred embodiment, the sweetener may include a at least one of glucose or sucrose, such as at least one of glucose syrup or cane sugar.

According to some embodiments, the sweetener may be present in an amount of at least 60 wt %, at least 65 wt %, at least 70 wt %, or at least 75 wt %, and no more than 80 wt %, no more than 85 wt %, no more than 90 wt %, or no more than 95 wt %, based on a total weight of the gummy formulation. In an embodiment in which the sweetener includes glucose and sucrose, a weight ratio of sucrose to glucose may be at least 0.15, at least 0.20, at least 0.25, or at least 0.30, and no more than 0.40, no more than 0.45, no more than 0.50, or no more than 0.55.

According to some embodiments, glucose may be present in an amount of at least 40 wt %, at least 45 wt %, at least 50 wt %, or at least 55 wt %, and no more than 60 wt %, no more than 65 wt %, no more than 70 wt %, or no more than 75 wt %, based on a total weight of the gummy formulation. According to some embodiments, sucrose may be present in an amount of at least 8 wt %, at least 10 wt %, at least 12 wt %, or at least 15 wt %, and no more than 20 wt %, no more than 25 wt %, no more than 30 wt %, or no more than 35 wt %, based on a total weight of the gummy formulation.

According to some aspects, inclusion of appropriate amounts of sugar plays an important role in the gelation of gelling agents such as pectin. In the presence of appropriate amounts of sugar and acidic conditions, pectin chains may undergo reduction of their affinity for water and the sugar may promote intermolecular hydrogen bonding. This facilitates the formation of a stable three-dimensional matrix or gel, as the pectin chains interact more closely. Sugar may also help lower the water activity, enhancing the strength and structure of the pectin matrix.

According to some aspects, the formulation may include an acid. According to some embodiments, the acid may include one or more of ascorbic acid, citric acid, tartaric acid, malic acid, lactic acid, acetic acid, or the like. According to a preferred embodiment, the acid may include citric acid. According to some embodiments, citric acid may be present in an amount of at least 0.50 wt %, at least 0.50 wt %, at least 0.70 wt %, or at least 0.9 wt %, and no more than 1.0 wt %, no more than 1.5 wt %, no more than 2.0 wt %, no more than 3.0 wt %, or no more than 5.0 wt %, based on a total weight of the gummy formulation. A weight ratio of acid to gelling agent may be at least 0.005, at least 0.10, at least 0.15, or at least 0.20, and no more than 0.40, no more than 0.50, no more than 0.60, or no more than 0.80.

Including the acid in appropriate amounts, e.g., those disclosed, may be important in forming a matrix, especially with pectin, because gelation may require a low pH (typically between 2.8 and 3.5). Acidic conditions reduce the negative charge on pectin's galacturonic acid residues, minimizing electrostatic repulsion and enabling hydrogen bonding and hydrophobic interactions between pectin chains. This promotes the formation of a stable gel network. Insufficient acid can result in poor gel formation and a weak matrix, while excessive acid can hinder gelation or affect product taste and stability. Proper acid concentration may ensure optimal gel strength, texture, and matrix integrity.

In some aspects, the excipients may also include a flavoring agent. The flavoring agent may include one or more of vanilla extract; synthetic vanillin; fruit essences and juices like strawberry (flavonoids), raspberry, orange, lemon, lime, and apple (various organic acids); chocolate flavoring agents such as cocoa solids and/or chocolate liquor; mint extracts containing menthol or synthetic equivalents; caramel flavors; nut extracts or oils like almond (benzaldehyde), hazelnut (amandin), and walnut (anisaldehyde); citric acid; and other known flavoring agents. In some embodiments, the flavoring agent consists of a natural flavoring agent. In a preferred embodiment, the flavoring agent contains a natural apple flavoring and does not include any synthetic flavoring agents.

According to some embodiments, the flavoring agent may be present in an amount of no more than 4 wt %, no more than 6 wt %, no more than 10 wt %, or no more than 12 wt %, based on a total weight of the gummy formulation.

In some aspects, the excipients may additionally include one or more of a colorant, a stabilizer, an emulsifier, and a preservative. The colorant may include natural and/or artificial colorants, such as Tartrazine (Yellow 5), Sunset Yellow FCF (Yellow 6), Allura Red AC (Red 40), Quinoline Yellow WS (Yellow 7), Carmoisine (Quinolone or Red 2), Amaranth (Red 2G), Erythrosine (Red 3), Indigo carmine (Blue No. 1), Brilliant Blue FCF (Blue 1), Green S (Green 3). Natural colorants may include one or more of beta-carotene (giving orange and yellow hues), anthocyanins (producing red, purple, and blue colors depending on pH levels), chlorophyllin (providing green shades), paprika extract (granting red tones), annatto extract (yielding yellow to red hues), turmeric root powder (offering brown coloration), beetroot juice, and spirulina.

According to some embodiments, the coloring agent may be present in an amount of no more than 0.5 wt %, no more than 0.7 wt %, no more than 1 wt %, or no more than 2 wt %, based on a total weight of the gummy formulation.

The stabilizer may include an antioxidant designed to preserve and reduce oxidative decay of the active ingredients. Such antioxidants may include vitamin C, vitamin E, beta-carotene, selenium, glutathione peroxidase, coenzyme Q10, resveratrol, curcumin, epigallocatechin gallate (EGCG), astaxanthin, and the like. Such antioxidants may provide a dual benefit in that they both stabilize the suforaphane and its precursors, as well as providing a direct benefit to a consumer.

According to some embodiments, the stabilizer may be present in an amount of no more than 0.5 wt %, no more than 0.7 wt %, no more than 1 wt %, or no more than 2 wt %, based on a total weight of the gummy formulation.

The emulsifier may include common food and drug grade emulsifiers known in the art, including but not limited to polysorbate 80, sodium lauryl sulfate, and Tween (polysorbate).

According to some embodiments, the emulsifier may be present in an amount of no more than 0.5 wt %, no more than 0.7 wt %, no more than 1 wt %, or no more than 2 wt %, based on a total weight of the gummy formulation.

The preservative may include common natural and synthetic food and drug grade preservatives known in the art, including but not limited to sodium citrate, sodium benzoate, sorbic acid, and calcium propionate. According to some preferred embodiments, the preservative may include sodium citrate.

According to some embodiments, the preservative may be present in an amount of no more than 0.5 wt %, no more than 0.7 wt %, no more than 1 wt %, or no more than 2 wt %, based on a total weight of the gummy formulation.

### Formulation

Conventionally, sulforaphane supplements have been provided in the form of pills or capsules. However, these types of formulations are undesirable and unsuitable for many patients. In particular, these formulations are unsuitable for pediatric patients and/or patients diagnosed with autism or a similar neurological disorder that causes particular sensitivity to sensory and physical stimuli, such as smell, taste, texture, mouthfeel, etc. These patients may be unable to tolerate dosing with a formulation the provides a poor sensory experience. Accordingly, existing forms of sulforaphane supplements (i.e. Tablets, capsules, etc.) may result in poor compliance among these patient groups or may even result in worsening of symptoms due to the need for regular exposure to aggravating sensory triggers.

The disclosed formulation solves this issue by providing a formulation that has a positive taste, smell, texture, and mouthfeel that is likely to encourage compliance with a dosing regimen in pediatric patients and/or patients with sensory processing difficulties. The disclosed formulations include gummy formulations, gummies, gummy candy type formulations, jellies, jelly candy type formulations, gum drops, jelly beans, and the like.

In the disclosed formulations, the active ingredients may be distributed and immobilized within a matrix. The matrix may have a three-dimensional structure formed by interconnected molecules of the gelling agent, defining a pore, including a plurality of pores. According to some aspects, the glucoraphanin, the myrosinase, and the sulforaphane are disposed within pores or interstitial spaces of the matrix and immobilized therein. This allows for glucoraphanin and myrosinase to be present within the gummy, without being free to move, contact one another, and react to form sulforaphane prior to ingestion by a patient. Instead, the immobilized glucoraphanin and myrosinase are only released to contact one another and react when the gummy is chewed by a patient. When the glucoraphanin and myrosinase come into contact during chewing, sulforaphane may then be formed in vivo, at the point of ingestion. This mimics the natural formation of sulforaphane from glucoraphanin and myrosinase which occurs during chewing of broccoli.

According to some embodiments, the formulation may not include capsules, microcapsules, or other form of encapsulation, e.g., especially to separate glucoraphanin and myrosinase. According to some embodiments, the glucoraphanin and myrosinase may be continuously distributed throughout the formulation structure and may not be confined to a particular area to separate them, such as within a microcapsule, a core, shell, or a coating.

According to some preferred embodiments, the gelling agent includes pectin. The matrix formed by pectin is a three-dimensional network of polysaccharide chains, primarily composed of galacturonic acid units linked via α-1,4 glycosidic bonds. During gelation, these chains interact through hydrogen bonds, hydrophobic interactions, and, in low-methoxyl pectin, ionic cross-linking via divalent cations (typically calcium ions). The resulting structure is a porous, hydrated gel matrix in which water and other molecules are trapped within the pores and interstitial spaces. This network may impart mechanical stability, control diffusion, and can encapsulate active ingredients or other substances within the gel. According to some aspects, the glucoraphanin, the myrosinase, and the sulforaphane are disposed within pores or interstitial spaces of the matrix and immobilized therein.

### Method of Treatment

The present disclosure also provides a method of treating an individual by administering one or more of the disclosed sulforaphane gummies one or more times per day. For example, the disclosed methods may include treating an individual by administering one gummy, twice per day, e.g. one gummy in the morning and one gummy in the evening.

In preferred embodiments, the individual may be a patient who has been diagnosed with a neurological disorder, including but not limited to autism, ASD, attention deficit disorder (ADD), or ADHD. In some embodiments, the patient may be a pediatric patient.

In alternative embodiments, the gummy may be administered to an individual who has not been diagnosed with any disease or disorder, or who has been diagnosed with a disease which is not of a neurological nature. In such embodiments, the method of treatment may be a method of supplementation designed to provide a range of potential health benefits related to sulforaphane, including but not limited to cancer protection, suppression of inflammation, blood sugar regulation, strengthening of the immune system and immune responses, protection against neurodegenerative diseases, improvement of heart health, and/or improvement of brain health.

Methods of supplementation include administering one or more of the disclosed sulforaphane gummies to an individual one or more times per day. For example, the disclosed methods may include treating an individual by administering one gummy, twice per day, e.g. One gummy in the morning and one gummy in the evening. Administration may be separated by time intervals of at least 6 hours, at least 8 hours, at least 10 hours, or at least 12 hours, and no more than 16 hours, no more than 14 hours, and no more than 12 hours.

According to the disclosed methods, the patient may be instructed orally or in writing to chew the gummy rather than to swallow it. Thus, according to the disclosed methods, myrosinase and glucoraphanin reaction may begin in the mouth, rather than in the digestive tract.

As described herein, the formulations can provide several significant advantages and benefits over other formulations for sulforaphane supplementation, currently available in the art. However, the recited advantages are not meant to be limiting in any way, as one skilled in the art will appreciate that other advantages may also be realized upon practicing the present disclosure. It will be appreciated, moreover, that other applications for the disclosed cans are also possible and considered to fall within the scope of the present disclosure.

Furthermore, those skilled in the relevant art will recognize that changes can be made to the described embodiments while still obtaining the beneficial results. It will also be apparent that some of the advantages and benefits of the described embodiments can be obtained by selecting some of the features of the embodiments without utilizing other features, and that features from one embodiment may be combined with features from other embodiments in any appropriate combination. For example, any individual or collective features of method embodiments may be applied to apparatus, product or system embodiments, and vice versa. Accordingly, those who work in the art will recognize that many modifications and adaptations to the embodiments described are possible and may even be desirable in certain circumstances, and are a part of the disclosure. Thus, the present disclosure is provided as an illustration of the principles of the embodiments and not in limitation thereof, since the scope of the invention is to be defined by the claims.

What is claimed is:

1. A supplement formulation, comprising:
an active ingredient consisting of sulforaphane, glucoraphanin, and myrosinase;
an excipient comprising pectin, a sugar comprising sucrose, citric acid, and water; and
a matrix formed by interconnected molecules of the pectin, defining a plurality of pores therebetween,
wherein the sulforaphane, the glucoraphanin, and the myrosinase are disposed within the pores and immobilized therein,
wherein in a weight ratio of the pectin to the water is within a range of 0.05 to 1.2,
wherein a weight ratio of the citric acid to the pectin is within a range of 0.005 to 0.80,
wherein the sucrose is present in an amount within a range of 8 wt % to 35 wt %, based on a total weight of the gummy formulation, and
wherein a weight ratio of the glucoraphanin to the pectin is within a range of 0.001 to 0.2.

2. The supplement formulation of claim 1, wherein the pectin is included in an amount within a range of 1.5 wt % and 14 wt %, based on the total weight of the gummy formulation.

3. The supplement formulation of claim 2, wherein the pectin is included in the amount within the range of 3 wt % to 8 wt %, based on the total weight of the gummy formulation.

4. The supplement formulation of claim 1, wherein the sulforaphane is included in an amount within a range of 0.001 wt % to 0.04 wt %, based on a total weight of the gummy formulation.

5. The supplement formulation of claim 4, wherein the sulforaphane is included in the amount within the range of 0.007 wt % to 0.02 wt %, based on a total weight of the gummy formulation.

6. The supplement formulation of claim 1, the sugar further comprising glucose.

7. The supplement formulation of claim 6, wherein a weight ratio of the sucrose to the glucose is within a range of 0.15 to 0.55.

8. A supplement formulation, comprising:
an active ingredient consisting of sulforaphane, glucoraphanin, and myrosinase, the formulation comprising a white mustard seed powder comprising the myrosinase;
an excipient comprising pectin, a sugar, citric acid, and water; and
a matrix formed by interconnected molecules of the pectin, defining a plurality of pores therebetween,
wherein the sulforaphane, the glucoraphanin, and the myrosinase are disposed within the pores and immobilized therein,
wherein a weight ratio of the white mustard seed powder to the glucoraphanin is within a range of 0.001 to 1,
wherein a weight ratio of the glucoraphanin to the pectin is within a range of 0.001 to 0.2, and
wherein a weight ratio of the white mustard seed powder to the pectin is within a range of 0.001 to 0.9.

9. The supplement formulation of claim 8, wherein the weight ratio of the white mustard seed powder to the glucoraphanin is within the range of 0.01 to 0.50.

10. The supplement formulation of claim 9, wherein the weight ratio of the white mustard seed powder to the glucoraphanin is within the range of 0.02 to 0.10.

11. The supplement formulation of claim 8, wherein the weight ratio of the white mustard seed powder to the pectin is within the range of 0.005 to 0.7.

12. The supplement formulation of claim 11, wherein the weight ratio of the white mustard seed powder to the pectin is within the range of 0.2 to 0.4.

13. The supplement formulation of claim 8, the weight ratio of the glucoraphanin to the pectin is within a range of 0.005 to 0.10.

14. The supplement formulation of claim 13, the weight ratio of the glucoraphanin to the pectin is within a range of 0.008 to 0.01.

15. A method of supplementation, comprising:
administering the supplement formulation of claim 1 to a user,
instructing the user to chew the supplement formulation,
initiating a reaction of the myrosinase and the glucoraphanin in the user's mouth.

16. The method of claim 15, wherein the user is a pediatric patient who has been diagnosed with a neurological disorder.

17. The method of treatment of claim 15, further comprising administering one unit of the supplement formulation, twice per day.

18. The method of treatment of claim 16, further comprising a first administration of one unit of the supplement formulation and a second administration of one unit of the supplement formulation, the first and second administration being separated by an interval within a range of 8 to 14 hours.
